(19) United States
(12) Patent Application Publication
(10) Pub. No.: US 2021/0038314 A1
(43) Pub. Date: Feb. 11, 2021

(54) Method and System for Personalized Treatment Planning in Ablation of Cancerous Tissue

(71) Applicant: phenoMapper, LLC, San Jose, CA (US)

(72) Inventor: Henky Wibowo, San Jose, CA (US)

(21) Appl. No.: 16/989,679

(22) Filed: Aug. 10, 2020

Related U.S. Application Data
(60) Provisional application No. 62/885,310, filed on Aug. 11, 2019.

(51) Int. Cl. A61B 34/10; A61B 18/04; A61B 34/00

(52) U.S. Cl. CPC A61B 34/10; A61B 18/04; A61B 2018/00577; A61B 2034/101; A61B 2034/105; A61B 34/25

(57) ABSTRACT

An ablation planning method for calculating an optimum dose to ablate a tumor in a patient comprises the steps of detecting the tumor (and optionally confirming the boundary of the tumor); segmenting the tumor and adjacent tissue structures; computing an ablation zone model based prior known information about the tissue and the ablation instrument; and extracting tumor parameters from the segmented tumor. The extracted tumor parameters can include a wide range of tumor properties that affect ablation including, without limitation, texture, density, shape, and heterogeneity. An ablation dose to yield an optimized ablation zone is calculated based on the extracted tumor properties and the ablation zone model. Related systems are also described.

### CROSS-REFERENCE TO RELATED APPLICATIONS

This claims priority to provisional application No. 62/885,310, filed Aug. 11, 2019, the entirety of which is incorporated by reference for all purposes.

### BACKGROUND

This invention relates to ablation, and more particularly to ablation planning for treatment of cancerous tissue.

Minimally invasive localized therapies, including radio frequency (RF), laser, cryo, High Focused Ultrasound, Irreversible Electroporation and microwave ablation delivered via percutaneous or bronchoscopic approaches, are increasingly being used clinically and offer the potential for curative treatment of patients who are not surgical candidates. Successful clinical delivery of minimally invasive ablation techniques is challenging because it involves accurate placement of the ablation applicator within the target site, creation of an adequate ablation zone that encompasses the targeted tumor and a surrounding margin of normal tissue, while limiting unintended thermal damage to adjacent critical structures. Treatment planning systems typically utilize only pre-operative medical images including CT data to identify the tumor and recommend ablation device applicator position. It is not uncommon, when modeling ablation based on this limited information, for the actual ablation to be inconsistent or not identical to the predicted ablation zone, namely, the ablation model. This is undesirable and an improved system and method is desired that overcomes the above mentioned challenges.

### SUMMARY OF THE INVENTION

An ablation planning method comprises the steps of detecting a tumor (and optionally confirming the boundary of the tumor); segmenting the tumor and adjacent tissue structures; extracting tumor parameters; and computing an ablation zone or model based on the extracted tumor parameters and other prior known information about the tissue and the ablation instrument. Extracting tumor parameters includes obtaining a wide range of tumor properties that affect ablation including, without limitation, texture, density, shape, and heterogeneity.

The ablation device sensitive parameters are linked to radiomics signatures that improve the result of the traditional ablation zone modeling techniques and lead to better ablation treatment outcomes.

In embodiments, a method predicts ablation zones of minimally invasive ablation devices by supplementing an ablation modeling of tissue ablation with ablation device sensitive radiomics features. Supplementing traditional ablation modeling algorithm/technique with ablation device sensitive radiomics features generates a more accurate and personalized ablation prediction that will lead to better treatment outcome.

In embodiments, inputs to the dose prediction model include the geometry of the target tumor, physical properties of the tissue, dimensions of the ablation device's probe/applicator, position of the probe/applicator within the lesion, and extracted tissue properties. The radiomics algorithm extracts properties of the targeted tumor's size and shape, as well as texture, from pre-operative CT images.

In embodiments, the shape, size, and texture data computed through 3D wavelets are employed as radiomics features for more accurate dose prediction.

In embodiments, a system is operable to model ablation and predict an ablation dose for tumor control.

This and other features, objects and advantages of embodiments of the present invention will become apparent from the detailed description to follow, together with the accompanying drawings.

### BRIEF DESCRIPTION OF THE DRAWINGS

FIG. 1 is a flow diagram showing an ablation planning method in accordance with an embodiment of the invention;

FIG. 2 is a flow diagram showing a tumor segmentation in accordance with an embodiment of the invention;

FIGS. 3A-3C are illustrations of lesions automatically detected in accordance with an embodiment of the invention;

FIG. 4 is an illustration of a preliminary or original heat transfer model computed for a lesion, ablation device, and the predicted ablation zone encompassing the lesion for 30 W input power;

FIG. 5 is an illustration of a heat transfer model computed for the lesion and ablation device shown in FIG. 4, and supplemented with additional extracted tumor property information in accordance with an embodiment of the invention; and

FIG. 6 is an illustration comparing the predicted ablation zone of FIG. 4 to that of FIG. 5 where the light shaded surface region represents the computational model from FIG. 4, and the dark shaded surface region represent computational model from FIG. 5.

### DETAILED DESCRIPTION OF THE INVENTION

It is to be understood that the embodiments of the invention described herein are not limited to particular variations set forth herein as various changes or modifications may be made to the embodiments of the invention described and equivalents may be substituted without departing from the spirit and scope of the embodiments of the invention. As will be apparent to those of skill in the art upon reading this disclosure, each of the individual embodiments described and illustrated herein has discrete components and features that may be readily separated from or combined with the features of any of the other several embodiments without departing from the scope or spirit of the embodiments of the present invention. In addition, many modifications may be made to adapt a particular situation, material, composition of matter, process, process act(s) or step(s) to the objective(s), spirit or scope of the embodiments of the present invention. All such modifications are intended to be within the scope of the claims made herein.

Moreover, while methods may be depicted in the drawings or described in the specification in a particular order, such methods need not be performed in the particular order shown or in sequential order, and that all methods need not be performed, to achieve desirable results. Other methods that are not depicted or described can be incorporated in the example methods and processes. For example, one or more additional methods can be performed before, after, simultaneously, or between any of the described methods. Further, the methods may be rearranged or reordered in other implementations. Also, the separation of various system components in the implementations described above should not be understood as requiring such separation in all implementations, and it should be understood that the described components and systems can generally be integrated together in a single product or packaged into multiple products. Additionally, other implementations are within the scope of this disclosure.

Conditional language, such as “can,” “could,” “might,” or “may,” unless specifically stated otherwise, or otherwise understood within the context as used, is generally intended to convey that certain embodiments include or do not include, certain features, elements, and/or steps. Thus, such conditional language is not generally intended to imply that features, elements, and/or steps are in any way required for one or more embodiments.

Reference to a singular item, includes the possibility that there are plural of the same items present. More specifically, as used herein and in the appended claims, the singular forms “a,” “an,” “said” and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

It will also be understood that, although the terms first, second, etc. may be used herein to describe various elements, these elements should not be limited by these terms. These terms are only used to distinguish one element from another. Thus, a first element could be termed a second element without departing from the teachings of the present invention.

Some embodiments have been described in connection with the accompanying drawings. The figures are drawn to scale, but such scale should not be limiting, since dimensions and proportions other than what are shown are contemplated and are within the scope of the disclosed inventions. Distances, angles, etc. are merely illustrative and do not necessarily bear an exact relationship to actual dimensions and layout of the devices illustrated. Components can be added, removed, and/or rearranged. Further, the disclosure herein of any particular feature, aspect, method, property, characteristic, quality, attribute, element, or the like in connection with various embodiments can be used in all other embodiments set forth herein except where such features are exclusive of one another. Additionally, it will be recognized that any methods described herein may be practiced using any device suitable for performing the recited steps.

While a number of embodiments and variations thereof have been described in detail, other modifications and methods of using the same will be apparent to those of skill in the art. Accordingly, it should be understood that various applications, modifications, materials, and substitutions can be made of equivalents without departing from the unique and inventive disclosure herein or the scope of the claims.

All existing subject matter mentioned herein (e.g., publications, patents, patent applications and hardware) is incorporated by reference herein in its entirety except insofar as the subject matter may conflict with that of the present invention (in which case what is present herein shall prevail).

Overview

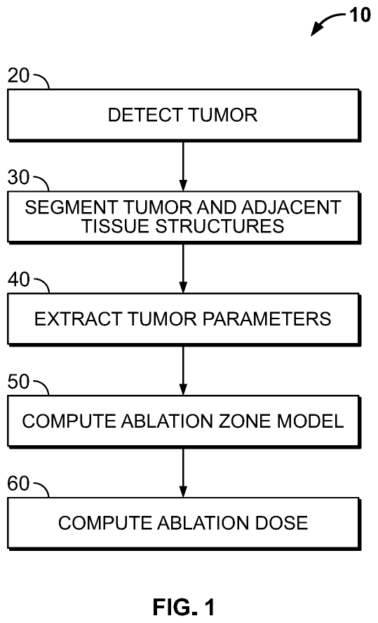

FIG. 1 shows an overview of a method 10 in accordance with embodiments of the invention to compute an ablation dose for optimizing ablation of a tumor in a patient. The method can be used for local tumor control. The steps shown in FIG. 1 comprise detecting a tumor (and optionally confirming the boundary of the tumor by the physician) 20; segmenting the tumor and adjacent tissue structures 30; extracting additional tumor parameters as described herein 40; computing an ablation zone or model 50, and calculating an ablation dose based on supplementing the ablation zone model with the additional extracted tumor parameters 60.

Tumor Detection

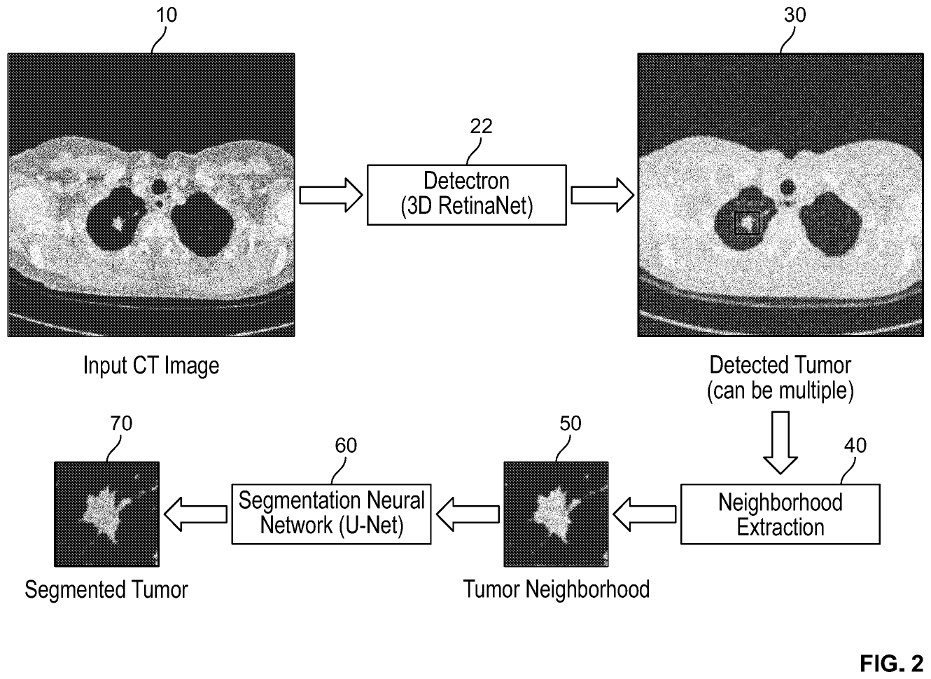

With reference to FIG. 2, a tumor is detected and segmented from a 3D image data set such as CT image data 10. Localization of the targeted tumor is executed through an object detection algorithm 22 such as a detectron and the tumors' neighborhood is then cropped 40 to create a sub-image focusing on tumors 50. Once the method obtains a sub-image, it carries out semantic segmentation 60 to segment a tumor 70 and extract radiomics features of the tumor, discussed herein.

An exemplary detectron is described in RetinaNet [9], but the parameters were adjusted as described herein in order to increase its sensitivity. The settings allow the method to detect more tumors and pre-compute radiomics features for each detected lesion.

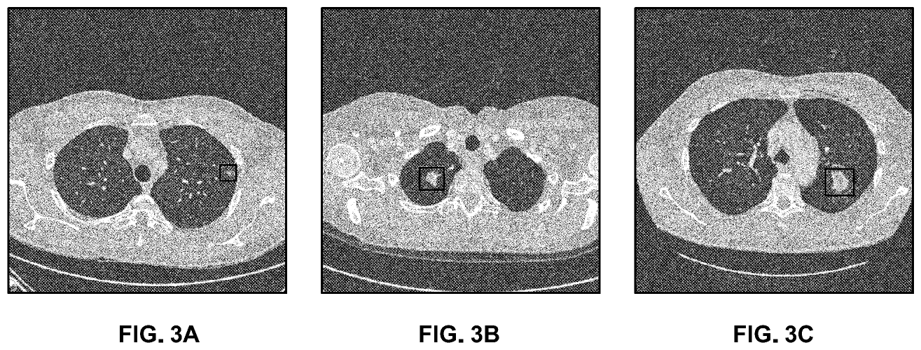

FIGS. 3A-3C illustrate lesions (or nodules) automatically detected by a method in accordance with the present invention. The detectron 22 could detect both small (e.g., FIG. 3A) and relatively large (e.g., FIG. 3C) lesions.

Segmentation

With reference again to FIG. 2, once a tumor is detected, a sub-image of its neighborhood will be created. In embodiments, when the segmentation method is based on U-Net or other similar models/methods, the size of a cross-section of a sub-image is fixed. In embodiments, a smaller sub-image is upscaled to a bigger one, but such upscaling should be carefully carried out because a very small lesion is vastly different in size than a large lesion. Consequently, upscaling a very small sub-image to a large one may result in excessive blurring and artifacts.

Another embodiment of the invention utilizes multiple CNN models such as, without limitation, the U-Net models trained for different image sizes [11]. When a bounding box of a suspect region is identified, it will be expanded to make a square region. Next, a sub-image is created and it will be up-scaled to match the closest size employed in a model. For example, in embodiments, three models are prepared with cross-section resolutions of 48×48, 96×96, and 198×198 pixels with voxel spacing 0.5 mm along the x-, y-, and z-axes. If a bounding box of a detected tumor is of size 36×32 pixels, it will be expanded to 36×36. Then, a sub-image is created with 36 transverse slices. Finally, in this embodiment, it will be up-scaled to 48×48×48.

With reference again to FIG. 2, in embodiments of the invention, a 3D CNN model such as the 3D U-Net-based model is employed for semantics segmentation 60 to extract a tumor as a region of interest (ROI). A bounding box fit to this ROI with a margin (e.g., 1 cm margin) is obtained 70 and a corresponding sub-image is created afterward. The method applies this ROI as a mask to exclude irrelevant regions when final texture statistics are to be computed. See also [14].

Extraction/Radiomics

Next, tumor features are extracted from the segmented tumor. In embodiments of the invention, several radiomics features can be extracted from medical imaging data such as CT data including density/intensity, size, shape, wavelet and texture analysis of the tumor, as well as heterogeneity index of the lesion and surrounding tissue. Extracted features may include subtlety, internal structure, calcification, sphericity, margin, lobulation, speculation, solidity, and malignancy.

In another embodiment, non-tumor type extracted radiomics are incorporated into the model and can include such features as the presence of blood vessels, tubular anatomy containing air or other substance that may become a heat sink to the thermal energy and affect the predicted ablation zone. However, it is to be understood that more or less radiomics features can be extracted and the invention is not intended to be so limited and except where recited in the appended claims.

In embodiments, texture is characterized. To obtain texture statistics, for example, a 3D Daubechies wavelet transform can be applied in the ROI sub-image to evaluate intratumor heterogeneity. The method utilizes results from wavelet transform to compute variance, entropy, and energy within the ROI. The complexity of tumor texture can be reflected by the entropy where a regular texture yields minimum entropy.

In embodiments, additional extracted properties of the ROI are acquired including: spiculation, shape, and density. These morphological properties, especially spiculation, are highly predictive of malignancy. See e.g., [12]. In embodiments of the invention, spiculation is roughly evaluated by difference between density of the central region and that of the margin. The shape property is evaluated by roundness of the ROI in the transverse view. The density is measured by the average of HU values within the ROI. In embodiments, these morphological properties are scaled from 0.0 to 1.0. These are comparative values among patients in a database. For instance, scaled spiculation with value of 1.0 means the largest density difference between the central and margin regions in a lesion database.

The morphological properties, size and texture analysis of the tumor can be summarized as a part of the tumor parameters or features which are input to the models described herein. Categories of microwave (or other energy modality) sensitive parameters can include tissue properties including electrical and heat transfer property, morphological shape (e.g., spiculated, elongated, round), water and fat contents, vascularity of the tumor, heterogeneity and homogeneity index of the tumor, compactness of the tumor and presence of anatomical structure that may act as heat sink in surrounding area around the lesion or ablation zone.

Compute Ablation Zone Model

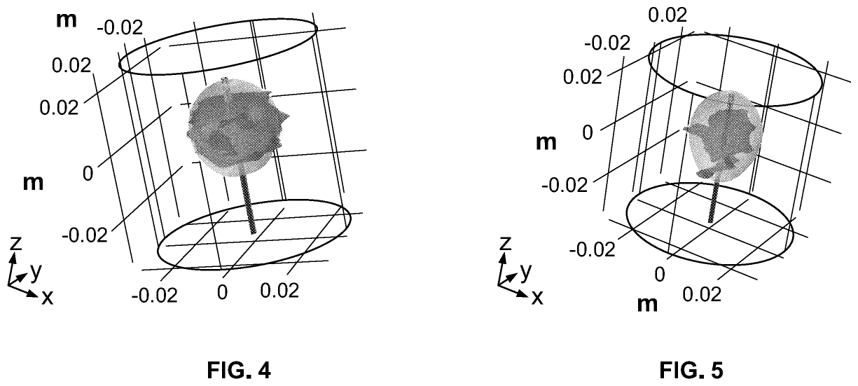

Next, and with reference to FIG. 4, an ablation zone model is provided or computed to generate an ablation zone given the instrument dimensions, tissue type and known tumor properties (e.g., size), and power setting (e.g., 30 W). An example of a heat transfer tool for predicting ablation zones is COMSOL HEAT TRANSFER MODULE, COMSOL Inc. (Los Altos, Calif.). See also [14]. However, other heat transfer tools may be used, and supplemented with the novel extracted data described herein.

Dose Calculation

Next, ablation dose is calculated. The ablation dose computation is based on supplementing the heat transfer model with the extracted radiomics features to yield an accurate ablation zone, and that results in minimum cancer recurrence rate and damage in surrounding normal tissue. In the case of a lung nodule, in embodiments, a desired margin is such that the overall ablation zone should be 1 cm larger (diameter) than the targeted tumor.

In addition to including known (or prior) information (such as geometry of the device and dielectric property of the tissue type), one or more of the above described extracted properties are incorporated into the heat transfer model to predict an improved or optimized ablation zone. The addition of the extracted energy sensitive parameters will alter the preliminary or initial ablation model (namely, the ablation zone calculated without considering these energy sensitive parameters). Indeed, and without intending to being bound to theory, it is expected that the addition of the radiomics features will improve the accuracy of the ablation or bioheat transfer model, and will consequently lead to better ablation treatment outcomes.

One example of combining heat transfer modeling with one or more radiomics features (such as tissue density, heterogeneity index of the lesion and surrounding tissue) is mapping the radiomics feature(s) to the tumor properties used in the ablation zone model.

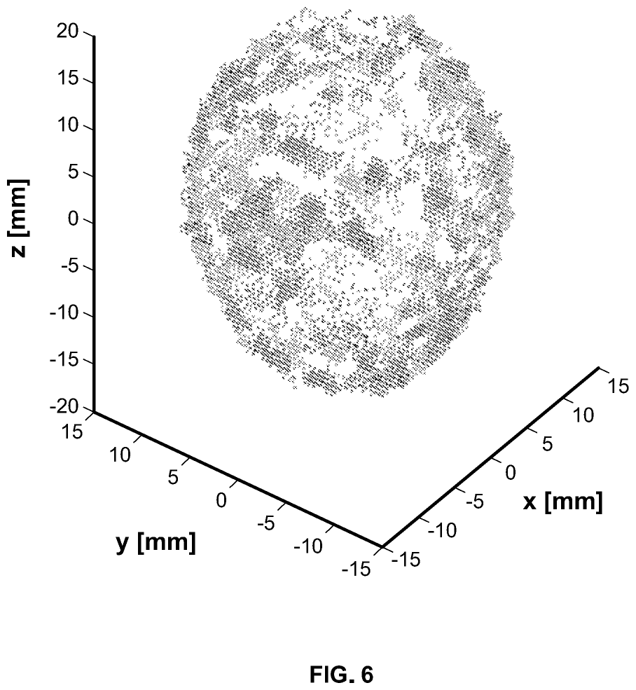

In another embodiment, and with reference to FIG. 5, combining the heat transfer modeling with the radiomic features is performed by including the radiomics features in the segmentation model (e.g., a trained CNN) to obtain a more accurate and detailed segmentation of the tumor vs. the preliminary/original model shown in FIG. 4. Consequently, the resulting heat transfer model shall be improved to the extent the tumor is more accurately defined. Indeed, and with reference to FIG. 6, a comparison of the ablation zones as predicted with 30 W input power of original and refined segmentation of a nodule is illustrated. The light/red shaded surface region represents the computational model from the original segmentation and the dark/blue shaded surface region represents computational model using the refined segmentation information. The Dice coefficient between the refined and original predicted ablation zone is 96% and the volume difference normalized to the volume of ablation of original segmentation is 8.02%. See also [14]. It follows that the predicted ablation dose shall be more accurate (more optimum) in view the more accurate segmentation of the tumor.

In another embodiment, the calculating step (or dose generation module) may be performed using machine learning algorithm such as trained neural network (e.g., a trained CNN mode). Non limiting examples of input features include: the preliminary ablation zone model, known device dimensions and relative location, known tumor properties, known adjacent tissue properties, and one or more extracted features from the tumor and adjacent tissue. A set of training data may include annotated 3D image data of tumors and adjacent tissue before and after ablation in which the above extracted properties are inputs/features to the model. The output can include an ablation dose to optimize ablation of the tumor.

Furthermore, the predicted ablation zones or ablation models described herein can be conveniently incorporated into a treatment planning system and/or image guidance system in a clinical setting. Since both treatment planning and image guidance system have been in use as part of the clinical workflow in interventional pulmonology or radiology, the addition of radiomics features analysis will not alter existing clinical workflow.

It is also to be understood that the method steps may be performed using a computer or processor programmed or operable to carry out the steps described herein. In embodiments of the invention, a system includes a processor, storage or memory, user interface devices, display, and communication interface to send and receive information to another whether wireless or otherwise. Additionally, a set of instructions can be stored on the memory device and executed by the processor.

Additionally, in embodiments, the processor can access and execute the following modules: localization module for identifying a tumor (e.g., detectron); segmentation module (e.g., U-Net) to segment the tumor and other adjacent tissue structures including, e.g., the vasculature, vessels and other structures in the vicinity of the tumor; radiomic or extraction algorithm to compute the energy-sensitive parameters (e.g., 3D Daubechies wavelet transform); a heat transfer module to determine/model the ablation zone (e.g., an FEA based algorithm such as COMSOL HEAT TRANSFER MODULE, COMSOL Inc. Los Altos, Calif.); and/or a dose generation module. Each module of the set can utilize one or more of: supervised learning (e.g., using logistic regression, using back propagation neural networks, using random forests, decision trees, etc.), unsupervised learning (e.g., using an Apriori algorithm, using K-means clustering), semi-supervised learning, reinforcement learning (e.g., using a Q-learning algorithm, using temporal difference learning), and any other suitable learning style. Examples of reinforcement learning include using a brute force approach, value function approach (e.g., Monte Carlo method, temporal difference method), direct policy approach, or otherwise reinforced. Examples of supervised learning include using: analytical learning, artificial neural network, backpropagation, boosting (meta-algorithm), Bayesian statistics, case-based reasoning, decision tree learning, inductive logic programming, Gaussian process regression, group method of data handling, kernel estimators, learning automata, minimum message length (decision trees, decision graphs, etc.), multilinear subspace learning, Naive Bayes classifier, nearest neighbor algorithm, probably approximately correct learning (pac) learning, ripple down rules, a knowledge acquisition methodology, symbolic machine learning algorithms, subsymbolic machine learning algorithms, support vector machines, minimum complexity machines (mcm), random forests, ensembles of classifiers, ordinal classification, data pre-processing, handling imbalanced datasets, statistical relational learning, or otherwise learned. Examples of unsupervised learning include using a method of moments approach or otherwise learned.

The invention may be used to compute ablation dose for a wide range of ablation-type devices including without limitation microwave, RF, cryo, ultrasound, laser, and radiation or radiotherapy.

In embodiments, the computed dose includes a power setting for an activation period. Exemplary power ranges from 60-100 Watts. Exemplary activation period ranges from 5-15 minutes. However, it is to be understood that the computed dose, power and activation time for the present invention may vary widely except as where recited in any appended claims.

Additionally, although the invention has particular use for planning ablation for lung tumors and nodules, the invention is not so limited. Examples of other tumor types for which ablation zone models and dose may be computed include, without limitation, lung, breast, liver, kidney, colon, prostrate, and ovarian.

### REFERENCES

Below is a list of publications, each of which is incorporated herein by reference in its entirety for all purposes:
[1] W. Zhu, C. Liu, W. Fan, and X. Xie, “DeepLung: Deep 3D Dual Path Nets for Automated Pulmonary Nodule Detection and Classification,” in 2018 IEEE Winter Conference on Applications of Computer Vision (WACV), 2018, pp. 673-681.
[2] R. J. Gillies, P. E. Kinahan, and H. Hricak, “Radiomics: Images Are More than Pictures, They Are Data,” Radiology, vol. 278, no. 2, pp. 563-577, February 2016.
[3] H. J. W. L. Aerts et al., “Decoding tumour phenotype by noninvasive imaging using a quantitative radiomics approach.”
[4] B. Lou et al., “An image-based deep learning framework for individualising radiotherapy dose: a retrospective analysis of outcome prediction,” Lancet Digit. Heal., vol. 1, no. 3, pp. e136-e147, July 2019.
[5] F. J. Wolf, D. J. Grand, J. T. Machan, T. A. DiPetrillo, W. W. Mayo-Smith, and D. E. Dupuy, “Microwave Ablation of Lung Malignancies: Effectiveness, CT Findings, and Safety in 50 Patients,” Radiology, vol. 247, no. 3, pp. 871-879, June 2008.
[6] E. S. Alexander, C. A. Hankins, J. T. Machan, T. T. Healey, and D. E. Dupuy, “Rib Fractures after Percutaneous Radiofrequency and Microwave Ablation of Lung Tumors: Incidence and Relevance,” Radiology, vol. 266, no. 3, pp. 971-978, March 2013.
[7] C. Chu, D. L. Belavy, G. Armbrecht, M. Bansmann, D. Felsenberg, and G. Zheng, “Fully Automatic Localization and Segmentation of 3D Vertebral Bodies from CT/MR Images via a Learning-Based Method,” PLoS One, vol. 10, no. 11, p. e0143327, 2015.
[8] X. Yang, Z. Zeng, and S. Yi, “Deep convolutional neural networks for automatic segmentation of left ventricle cavity from cardiac magnetic resonance images,” IET Comput. Vis., vol. 11, no. 8, pp. 643-649, December 2017.
[9] T. Y. Lin, P. Goyal, R. Girshick, K. He, and P. Dollar, “Focal Loss for Dense Object Detection,” in Proceedings of the IEEE International Conference on Computer Vision, 2017, vol. 2017-Octob, pp. 2999-3007.
[10] S. G. Armato et al., “LUNGx Challenge for computerized lung nodule classification,” J. Med. Imaging, vol. 3, no. 4, p. 044506, December 2016.
[11] O. Ronneberger, P. Fischer, and T. Brox, “U-net: Convolutional networks for biomedical image segmentation,” in Lecture Notes in Computer Science (including subseries Lecture Notes in Artificial Intelligence and Lecture Notes in Bioinformatics), vol. 9351, Springer, Cham, 2015, pp. 234-241.
[12] A. Snoeckx et al., “Evaluation of the solitary pulmonary nodule: size matters, but do not ignore the power of morphology,” Insights Imaging, vol. 9, no. 1, p. 73, February 2018.
[13] G. Deshazer et al., “Physical modeling of microwave ablation zone clinical margin variance”, Medical Physics, vol. 43, No. 4, April 2016.
[14] P. Taeprasartsit et al., “A personalized approach for microwave ablation treatment planning fusing radiomics and bioheat transfer modeling”, Proc. SPIE 11315, Medical Imaging 2020: Image-Guided Procedures, Robotic Interventions, and Modeling, 1131532 (16 Mar. 2020).

1. An ablation planning method for ablating a tumor within an anatomy of a patient, the method comprising:
a) detecting the tumor from 3D image data of the anatomy;
b) segmenting the tumor and adjacent structures in the vicinity of the tumor from the 3D image data of the anatomy;
c) computing a preliminary ablation zone model based on device characteristics of a candidate medical device;
d) extracting a plurality of tumor properties from the segmented tumor; and
e) calculating an ablation dose yielding an ablation zone encompassing the tumor based on the preliminary ablation zone model and the extracted tumor properties.

2. The method of claim 1, further comprising receiving multiple digital images of the 3D image data of the anatomy of the patient.

3. The method of claim 1, wherein the computing step is performed by a heat transfer model, and the calculating step is performed by adjusting the first heat transfer model based on the extracted tumor properties.

4. The method of claim 1, further comprising extracting adjacent tissue properties from the segmented adjacent structures, and the calculating step is further based on the extracted adjacent tissue properties.

5. The method of claim 1, wherein the detecting step is performed with a trained CNN.

6. The method of claim 1, further comprising confirming or adjusting a boundary of the tumor.

7. The method of claim 1, wherein step (c) is performed prior to step (d), or carried out simultaneously.

8. The method of claim 1, wherein the tumor properties are selected from the group consisting of texture, size, shape, and density.

9. The method of claim 1, wherein the candidate medical device characteristics are selected from the group consisting of power and device geometry.

10. The method of claim 4, wherein the segmented adjacent structures are selected from the group consisting of airways and vessels.

11. An ablation planning system for maximizing local tumor control within an anatomy of a patient, the system comprising a memory, one or more user input devices, and a processor wherein the processor is programmed with a set of instructions to:
a) detect and segment the tumor and adjacent structures in the vicinity of the tumor from 3D image data of the anatomy;
b) compute a preliminary ablation zone model based on device characteristics of a candidate medical device and known information;
c) extract a plurality of tumor properties from the segmented tumor; and
d) calculate an ablation dose yielding an ablation zone based on the preliminary ablation zone model and the extracted tumor properties.

12. The system of claim 11, further comprising a communication interface for receiving multiple digital images of the 3D image data of the anatomy of the patient.

13. The system of claim 11, wherein the processor is operable to compute the preliminary ablation zone model using a heat transfer model, and to calculate the ablation dose by adjusting the first heat transfer model based on the extracted tumor properties.

14. The system of claim 11, wherein the processor is further operable to extract a plurality of adjacent structure properties from the segmented adjacent structures.

15. The system of claim 14, wherein the processor is operable to detect using a trained CNN.

16. The system of claim 11, wherein the processor is operable to confirm or adjust a boundary of the tumor based on physician instructions received via the user input device.

17. The system of claim 11, wherein to extract a plurality of tumor properties from the segmented tumor comprises extracting tumor properties selected from the group consisting of texture, size, shape, and density.

18. The system of claim 11, wherein to compute a preliminary ablation zone model based on device characteristics of a candidate medical device comprises selecting medical device characteristics from the group consisting of power and device geometry.

19. The system of claim 14, wherein to extract a plurality of adjacent structure properties from the segmented adjacent structures comprises extracting one or more of the following properties: type, shape, thermal conductivity, thermal effusivity, and heat capacity.

20. An ablation planning method for ablating a tumor within an anatomy of a patient, the method comprising:
a) detecting the tumor from 3D image data of the anatomy;
b) segmenting the tumor and adjacent structures in the vicinity of the tumor from the 3D image data of the anatomy;
c) extracting a plurality of tumor properties from the segmented tumor; and
d) calculating an ablation dose yielding an ablation zone encompassing the tumor and based on prior known tissue properties, device characteristics, device location, and the extracted tumor properties, and
wherein the calculating step is performed using a trained machine learning model.
